(19) United States
(12) Patent Application Publication
(10) Pub. No.: US 2007/0077315 A1
(43) Pub. Date: Apr. 5, 2007

(54) Child discipline composition and method

(76) Inventor: Shauna M. Schoenborn, Steelville, MO (US)

Correspondence Address: WEISS & MOY PC, 4204 NORTH BROWN AVENUE, SCOTTSDALE, AZ, 85251, US

(21) Appl. No.: 11/241,055

(22) Filed: Sep. 30, 2005

(51) Int. Cl. A61K 36/73; A61K 36/71

(52) U.S. Cl. 424/726; 424/765

(57) ABSTRACT

A composition for substantially deterring unwanted behaviors in children is provided. The composition comprises water, goldenseal tincture, and apple cider vinegar in an effective amount. The composition is sprayed toward the mouth of the child after the child engages in the unwanted behavior. The composition may be sprayed from an aerosol can, sprayer, or the like.

### FIELD OF THE INVENTION

This invention relates generally to a composition and method for behavior modification and more particularly to a composition and method for disciplining a child.

### BACKGROUND OF THE INVENTION

Child discipline is an important and emotional issue for parents. Parents worry about their child's misbehavior and how they should handle it. They become angry and frustrated with ongoing behavior problems, but dislike having to punish their children. Effective discipline helps children develop empathy and self-control while too harsh of discipline puts a child at risk for numerous emotional and behavioral problems.

Throughout history, there have been a number of ways suggested to discipline children, e.g. restrictions, spankings, time outs, etc. These and other techniques are generally used when the child's misbehavior is related to the use of their mouth: talking back to adults, lying, biting someone, swearing, spitting, refusing to eat, etc. Such other techniques include, for example, “Hot Saucing.” “Hot Saucing” (a.k.a. “Hot Tongue”) involves placing Tabasco® hot sauce or a similar hot sauce on a child's tongue as a punishment for unacceptable behavior. Variations on the punishment include the use of lemon juice, soap or some other highly noxious substance. The sting is a memory that lingers so that the next time the child misbehaves, he or she may actually have some self-control and stop before misbehaving. Some people attest to the efficiency of such discipline techniques. Part of their appeal is they are immediate, but over within minutes, while grounding or the restriction of privileges requires parental oversight over time to ensure compliance. Unfortunately, the use of hot sauce and these other substances on children is dangerous because of the risk of swelling and/or irritation and the possibility of triggering unknown allergies and creating a potential choking hazard. In addition, a parent willing to try this discipline technique must often hold down the child or be forceful enough to enable one person to introduce the substance into another person's mouth. This often cannot be done so easily without inflicting physical injury, especially with a larger child and may teach the child fear, rather than respect.

Accordingly, there is a need for a substantially safe, harmless and effective composition and method for safely disciplining a child without the need for force or intimidation and which is easy to use with substantially immediate results. The present invention fulfills these needs and provides other related advantages.

### SUMMARY OF THE INVENTION

In accordance with this invention, the composition comprises about 65 to about 75 percent water, about 20 percent to about 35 percent pure apple cider vinegar, and about 2 to about 5 percent goldenseal tincture [1:2 concentration] In a preferred form, the composition comprises about 69.1 percent water, 27.5 percent apple cider vinegar and about 3.4 percent goldenseal tincture.

The composition may be administered by spraying the composition toward the misbehaving child's mouth to substantially deter the unwanted behavior, typically those unwanted behaviors having to do with the mouth. The composition may be administered from an aerosol can, sprayer or the like in an effective amount of one ounce or less.

Other features and advantages of the present invention will become apparent from the following more detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is an operational view of a child and aerosol can in accordance with the first embodiment of the invention, illustrating the composition sprayed into the mouth of the child; and

FIG. 2 is a perspective view of another embodiment of the container for administering the composition, illustrating a spray bottle.

### DETAILED DESCRIPTION OF THE PREFERRED EMBODIMENTS

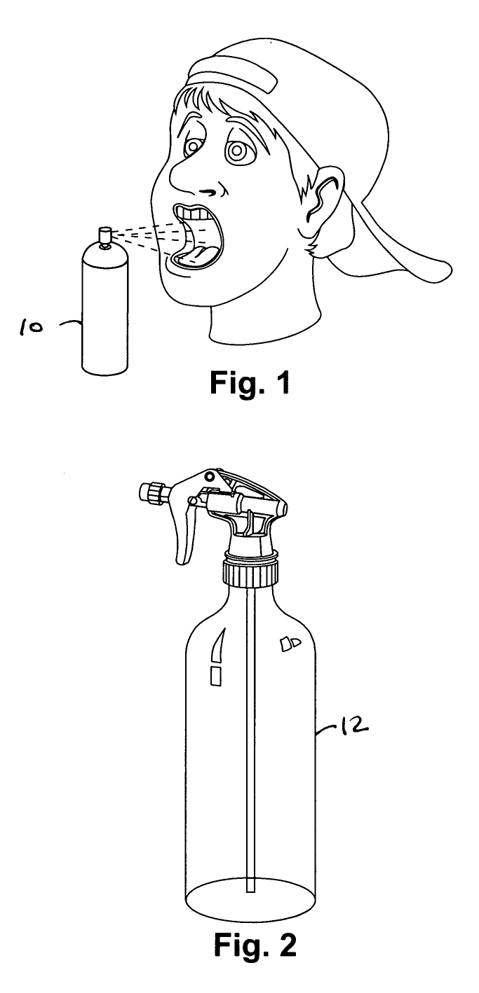

As shown in the drawings for purposes of illustration, the present invention is concerned with a novel composition and method for substantially discouraging unwanted behavior in children. In accordance with the present invention, and as illustrated with respect to a preferred embodiment in FIGS. 1-2, the composition is substantially harmless and environmentally friendly.

In accordance with this invention, the composition comprises about 65 to about 75 percent water, about 20 percent to about 35 percent pure apple cider vinegar, and about 2 to about 5 percent goldenseal tincture [1:2 concentration] In a preferred form, the composition comprises about 69.1 percent water, 27.5 percent pure apple cider vinegar and about 3.4 percent goldenseal tincture. Goldenseal tincture is available commercially as “Extract of Goldenseal” from Teetercreek Herbs, Route 5, Ava, Miss. 65508 as well as other herbal suppliers. The “Extract of Goldenseal” is made by goldenseal root in distilled water and pure gram alcohol to make a 1:2 extract. Goldenseal is also known by the names Yellow Root, Orange Root, Eye Root, Indian Turmeric, and Jaundice Root. Its official Latin name is Hydrastis Canadensis. As used herein, “tincture” means an alcoholic solution of a medicinal substance. Glycerin in place of alcohol may be used as an alcohol-free alternative to the tincture to create a “glycerite”, but its use is limited due to the sweetness of the glycerin which may not serve the desired deterrent effect on the child.

A preferred pure Apple Cider Vinegar is available as Braggs Organic from Live Food Products in Santa Barbara, Calif. The apple cider vinegar has a table strength of 5% acidity.

The preferred composition using goldenseal tincture may be prepared by the following process:

Boil 1-1¼ cup water.

Mix 1 tablespoon goldenseal tincture into the boiling water.

Turn off heat as the alcohol in the tincture quickly burns off.

Add ½ cup apple cider vinegar.

Cool before administration.

The mixture will fill about 12-14 1 ounce spray bottles.

Although the use of goldenseal tincture in the composition has been described, it is to be appreciated that other forms of goldenseal may be used within the confines of the invention by one skilled in the art. For example, a goldenseal infusion or decoction may be prepared by known methods by one skilled in the art in the amount of one teaspoon of powdered goldenseal root or two teaspoons of dried goldenseal root to one cup of water. As used herein, the term “decoction” means a water extract of bark or roots prepared at a low boil for 10-20 minutes; stronger than an infusion. “Infusion” means a composition made by steeping plants or plant extracts in hot water for 10-20 minutes. After the goldenseal infusion or decoction has been made, ½ cup of the apple cider vinegar may be added to the infusion or decoction.

The composition may be administered from a spray can 10 as shown in FIG. 1 or from a spray bottle 12 (FIG. 2) or the like. Aerosol administration of the composition is preferred because it makes it easier to direct the composition toward the mouth without having to physically restrain the child.

The method for deterring unwanted behaviors in a child using the composition comprises spraying an effective amount of a composition toward the mouth of the child after the child engages in the unwanted behavior, wherein said composition comprises water, goldenseal and vinegar; and repeating said spraying step a second time if the unwanted behavior continues after the initial spraying step.

In using the composition, if the child is behaving badly enough to warrant administration, walk up calmly to the child. The can or spray bottle containing the composition should be shaken well before administration. The composition may be administered without threatening the child or giving the child a warning and then counting to three or five (depending on the circumstance and child's age). The composition may be administered in doses of one ounce or less.

For example, a typical warning might be as follows: “Stop throwing your fit and yelling right now, if you don't stop I am going to spray you. Okay, I'm going to count to 3, and if you don't stop, you are making the choice to get sprayed.” If you tell a child he is in control of the situation and he is deciding on if he is going to stop the fit or not, it will sometimes make him feel more in control. Attempt to spray the child's mouth discreetly so that you have the best opportunity to get it into the mouth. As they are misbehaving is a good time to do so. Spraying across a closed mouth and onto the lips may still be unpleasant and effective as the composition is substantially bitter and will linger on their lips.

After administration, the child should be told why they got sprayed and what kind of behavior is expected from them. Do not laugh when you using the composition. This will teach children that being in trouble is funny or that the behavior is acceptable. The composition works well for unwanted behaviors such as biting, swearing, talking back/“sassing”, bad attitudes and yelling/screaming. Once the child calms down, apologizes and acts respectful, allow them to drink water. The taste will linger for a few minutes, and they will not want a repeat dose.

Certain precautions should be adopted when using the composition. The composition should not be used excessively and not on children under three years old. The child should not be held down in an attempt to spray the composition into their mouths. If the composition gets into the eyes, it may sting but will not cause harm. The eyes should be flushed with water if this occurs by accident. The composition should never intentionally be sprayed into the eyes.

From the foregoing, it is to be appreciated that the composition was specifically designed with a child's health and safety in mind and is substantially effective at effective deterring unwanted behaviors without harming the child.

Although a particular embodiment of the invention has been described in detail for purposes of illustration, various modifications may be made without departing from the spirit and scope of the invention. Accordingly, the invention is not to be limited, except as by the appended claims.

I claim:

1. A composition for substantially deterring unwanted behaviors in children, comprising:
about 65 to about 75 percent water, about 20 percent to about 35 percent pure apple cider vinegar, and about 2 to about 5 percent goldenseal.

2. The composition of claim 1, comprising about 69.1 percent water, 27.5 percent pure apple cider vinegar and about 3.4 percent goldenseal.

3. The composition of claim 2, wherein the goldenseal comprises goldenseal tincture.

4. The composition of claim 3, wherein the goldenseal tincture is comprised of one part goldenseal to two parts alcohol.

5. The method of making the composition of claim 3, comprising mixing about 1 tablespoon of goldenseal tincture into about 1-1¼ cup water, and adding about ½ cup apple cider vinegar.

6. A method for deterring unwanted behaviors in a child, comprising the steps of:
Spraying an effective amount of a composition toward the mouth of the child after the child first engages in the unwanted behavior;
Wherein said composition comprises water, goldenseal and vinegar; and
Repeating said spraying step a second time if the unwanted behavior continues after the initial spraying step.

7. The method of claim 6, wherein said effective amount comprises one ounce or less of said composition.

8. The method of claim 6, wherein said composition comprises about 65 to about 75 percent water, about 20 percent to about 35 percent pure apple cider vinegar, and about 2 to about 4.5 percent goldenseal.

9. The method of claim 8, wherein said composition comprises about 69.1 percent water, 27.5 percent pure apple cider vinegar and about 3.4 percent goldenseal.

10. The method of claim 9, wherein said goldenseal comprises goldenseal tincture.

11. The method of claim 6, wherein said composition is sprayed from an aerosol can.

12. The method of claim 6, wherein said composition is sprayed from a sprayer.
